(19) United States
(12) Patent Application Publication
(10) Pub. No.: US 2025/0257313 A1
(43) Pub. Date: Aug. 14, 2025

(54) CULTURE METHOD AND CULTURE DEVICE

(71) Applicant: HONDA MOTOR CO., LTD., Minato-ku, Tokyo (JP)

(72) Inventors: Minoru Goto, Wako-shi, Saitama-ken (JP); Nozomi Shiobara, Wako-shi, Saitama-ken (JP); Shohei Kinoshita, Wako-shi, Saitama-ken (JP); Mizuho Doi, Wako-shi, Saitama-ken (JP); Kenji Machida, Wako-shi, Saitama-ken (JP); Satoshi Shiozaki, Wako-shi, Saitama-ken (JP)

(21) Appl. No.: 18/851,107

(22) PCT Filed: Feb. 20, 2023
(86) PCT No.: PCT/JP2023/006053

§ 371 Date: Sep. 26, 2024

(30) Foreign Application Priority Data
Mar. 30, 2022 (JP) 2022-055036

(51) Int. Cl. C12N 1/12; C12M 1/00; C12M 1/34

(52) U.S. Cl. CPC C12N 1/12; C12M 29/20; C12M 41/06; C12M 41/12

(57) ABSTRACT

A culture device comprises: a gas supply unit; and a plurality of containers. The gas supply unit is connected with the plurality of containers through a plurality of gas supply lines, respectively. The plurality of gas supply lines are provided with a plurality of valves, respectively. While microalgae are cultured in the plurality of containers, the opening degrees of the plurality of valves are adjusted. By such adjustment of the opening degrees, a greater amount of gas is supplied to one of the plurality of containers than those of the other containers.

### TECHNICAL FIELD

The present invention relates to a culture method and a culture device for culturing microalgae in a culture solution.

### BACKGROUND ART

Heretofore, efforts aimed at mitigating climate change or reducing its impact have continued, and toward the realization thereof, research and development in relation to the reduction of carbon dioxide emissions are being carried out. From this point of view, attention has been focused on microalgae. This is because microalgae consume carbon dioxide through photosynthesis. Accordingly, a culture device for culturing microalgae is anticipated as a device that contributes to mitigating climate change or reducing its impact.

A culture solution and microalgae are accommodated in an accommodation unit. A sufficient amount of a culturing gas used in culturing for carrying out photosynthesis of the microalgae is supplied to the accommodation unit. Although the culture solution and the microalgae are subjected to diffusion to some extent by the culturing gas, the microalgae undergo settling or sedimentation within the culture solution. When the microalgae undergo settling, the amount of light received by the microalgae decreases. As a result, the amount of photosynthesis becomes inadequate. From this standpoint, in the related art disclosed in JP 2018-537948 A, a gas used to prevent settling is intermittently supplied to the culture solution inside the accommodation unit. In this related art, the gas used to prevent settling is supplied to the culture solution once every 30 minutes, and thereby causes the microalgae that have settled to float or be suspended within the culture solution.

### SUMMARY OF THE INVENTION

It is assumed that microalgae are simultaneously cultured in a plurality of accommodation units. In this case, a large amount of microalgae can be cultured, and thus a large amount of carbon dioxide can be expected to be consumed.

In this case, the gas used to prevent settling is supplied to the plurality of accommodation units. In this configuration, when the gas used to prevent settling is simultaneously supplied to all the accommodation units, then as shown in FIG. 4, valves respectively provided in the plurality of accommodation units are simultaneously opened, and a large amount of the gas used to prevent settling is temporarily supplied from a gas supply unit. For this reason, in spite of the fact that the gas used to prevent settling is supplied intermittently, the gas supply unit must have an ability to simultaneously supply a large amount of the gas used to prevent settling.

A gas supply unit having such a capacity consumes a large amount of electric power per unit time period. Accordingly, in performing culturing in this manner, it is not easy to reduce the running cost.

The present invention has the object of solving the aforementioned problem.

According to one aspect of the present invention, there is provided a culture method for culturing microalgae in a culture device including a plurality of accommodation units configured to accommodate a culture solution and the microalgae, a gas supply unit configured to supply a gas to the plurality of accommodation units, and a plurality of gas supply lines configured to connect the gas supply unit with the plurality of accommodation units, respectively, wherein the plurality of gas supply lines are provided with a plurality of valves, respectively, the culture method comprising: an opening degree adjustment step of adjusting an opening degree of at least one of the plurality of valves during culturing of the microalgae in the plurality of accommodation units; and an agitation step of culturing the microalgae in a state in which the opening degree of the at least one of the plurality of valves is adjusted, wherein, in the opening degree adjustment step, by adjusting the opening degree of the at least one of the plurality of valves, supply of the gas to at least one accommodation unit of the plurality of accommodation units is stopped or a supply flow rate of the gas supplied to the at least one accommodation unit is decreased, and a supply flow rate of the gas supplied to at least one remaining accommodation unit is increased.

According to another aspect of the present invention, there is provided a culture device that cultures microalgae in a culture solution, the culture device comprising: a plurality of accommodation units configured to accommodate the culture solution and the microalgae; a gas supply unit configured to supply a gas to the plurality of accommodation units; a plurality of gas supply lines configured to connect the gas supply unit with the plurality of accommodation units, respectively; a plurality of valves provided in the plurality of gas supply lines, respectively; and a control unit configured to individually adjust an opening degree of each of the plurality of valves, wherein, by adjusting the opening degree of at least one of the plurality of valves during culturing of the microalgae in the plurality of accommodation units, the control unit stops supply of the gas to at least one accommodation unit of the plurality of accommodation units or decreases a supply flow rate of the gas supplied to the at least one accommodation unit, and increases a supply flow rate of the gas supplied to at least one remaining accommodation unit.

By adjusting the opening degree, the supply of the gas is stopped or the supply flow rate is decreased in at least one of the plurality of accommodation units. In contrast, the supply flow rate of the gas is increased in at least one remaining accommodation unit. That is, the supply flow rate of the gas is adjusted.

Therefore, even if the supply flow rate of the gas from the gas supply unit is constant, a large amount of gas can be temporarily supplied to at least one of the plurality of accommodation units. Therefore, it is not necessary to select a gas supply unit having an excessive capacity. In this instance, the excessive capacity is a capacity capable of simultaneously supplying, to the plurality of accommodation units, a gas for agitation in addition to a gas supplied in a general step.

That is, according to the present invention, it is possible to use a gas supply unit that has an appropriate capacity and consumes a small amount of electric power per unit time period. Therefore, in performing culturing, it is possible to reduce the running cost.

In the accommodation unit in which the supply flow rate of the gas is temporarily increased, the microalgae and the culture solution are agitated. Therefore, settling or flocculation of the microalgae is suppressed. Accordingly, the light is incident on the microalgae substantially evenly. Further, since the culture solution is also agitated, carbon dioxide becomes diffused throughout the entirety of the accommodation unit. Due to this reason, the microalgae actively carry out photosynthesis. Since a large amount of carbon dioxide becomes fixed in the microalgae by way of photosynthesis, such a feature can be expected to contribute to mitigating climate change or reducing its impact.

### BRIEF DESCRIPTION OF THE DRAWINGS

FIG. 1 is a schematic system diagram of a culture device according to an embodiment of the present invention;

FIG. 2 is a schematic process flow diagram of a culture method according to an embodiment of the present invention;

FIG. 3 is a graph showing the opening degrees of valves provided in first to third accommodation units during culturing, and changes in the gas supply flow rates according to the opening degrees of the valves; and

FIG. 4 is a graph showing changes in the opening degrees of valves provided in a plurality of accommodation units and changes in the gas supply flow rates according to the opening degrees of the valves in the related art.

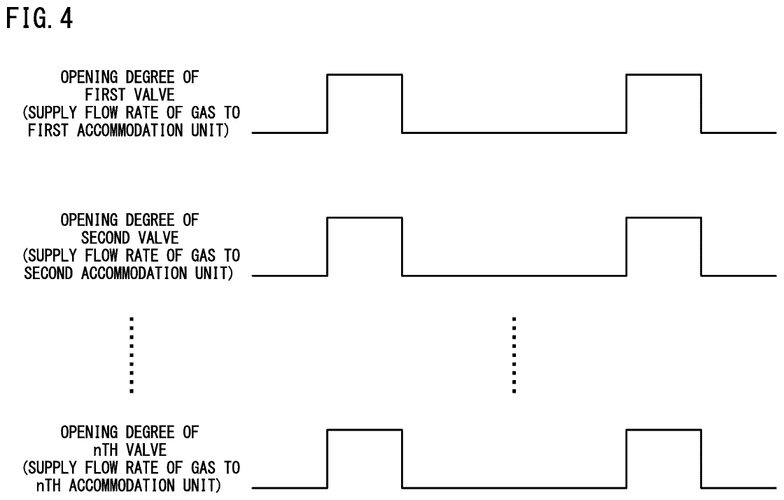

### DETAILED DESCRIPTION OF THE INVENTION

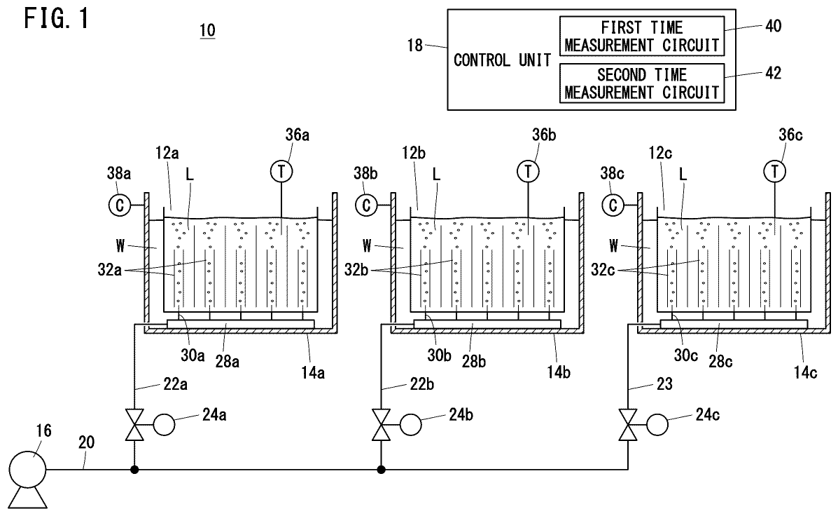

FIG. 1 is a schematic system diagram of a culture device 10 according to a present embodiment. The culture device 10 includes a plurality of accommodation units. In a first embodiment, for ease of understanding, a case where the number of accommodation units is three will be described as an example. Further, in order to easily distinguish the three accommodation units, the accommodation unit located on the leftmost side in FIG. 1 is referred to as a first accommodation unit 12a. In FIG. 1, the accommodation unit adjacent to the first accommodation unit 12a on the right side is referred to as a second accommodation unit 12b. The accommodation unit located on the rightmost side in FIG. 1 is referred to as a third accommodation unit 12c. The first accommodation unit 12a, the second accommodation unit 12b, and the third accommodation unit 12c have the same shape and volume.

A culture solution L and microalgae are accommodated in each of the first accommodation unit 12a to the third accommodation unit 12c. As understood from the above, the first accommodation unit 12a to the third accommodation unit 12c are culturing tanks for culturing microalgae. The culture solution L is typically water. Phosphorus, nitrogen, potassium, and the like are preferably added in advance to the culture solution L.

The first accommodation unit 12a, the second accommodation unit 12b, and the third accommodation unit 12c are accommodated in a first water storage unit 14a, a second water storage unit 14b, and a third water storage unit 14c, respectively. The first water storage unit 14a, the second water storage unit 14b, and the third water storage unit 14c are each formed of a material that transmits light. Water W is stored in each of the first water storage unit 14a, the second water storage unit 14b, and the third water storage unit 14c. The water W serves as a cooling medium for cooling the culture solution L inside the first accommodation unit 12a to the third accommodation unit 12c.

The culture device 10 includes an air pump 16 serving as a gas supply unit. The air pump 16 draws in and compresses the atmospheric air. The air pump 16 is electrically connected to a control unit 18.

An upstream end portion of a flow pipe 20 is connected to the air pump 16. The flow pipe 20 extends toward the third accommodation unit 12c. A first branch pipe 22a and a second branch pipe 22b are provided in the extending flow pipe 20. The first branch pipe 22a extends toward the first accommodation unit 12a, and the second branch pipe 22b extends toward the second accommodation unit 12b. Each of the first branch pipe 22a, the second branch pipe 22b, and a downstream end portion 23 of the flow pipe 20 is a gas supply line. In other words, the culture device 10 includes a plurality of (three in the present embodiment) gas supply lines.

The first branch pipe 22a is provided with a first solenoid valve 24a. The second branch pipe 22b is provided with a second solenoid valve 24b, and the downstream end portion 23 of the flow pipe 20 is provided with a third solenoid valve 24c. The first solenoid valve 24a, the second solenoid valve 24b, and the third solenoid valve 24c are electrically connected to the control unit 18. That is, the first solenoid valve 24a, the second solenoid valve 24b, and the third solenoid valve 24c are opened and closed in response to command signals from the control unit 18. The control unit 18 can set the first solenoid valve 24a, the second solenoid valve 24b, and the third solenoid valve 24c to any opening degree between fully opening and fully closing.

A first main pipe 28a is provided at the bottom of the first water storage unit 14a. The first branch pipe 22a is connected to the first main pipe 28a. The first main pipe 28a extends along the horizontal direction at the bottom of the first water storage unit 14a. A plurality of first auxiliary pipes 30a branch from the first main pipe 28a. The first auxiliary pipes 30a extend along a substantially vertical direction.

A plurality of first guide members 32a are provided inside the first accommodation unit 12a. The first guide members 32a are disposed above the first auxiliary pipes 30a, respectively, and receive a gas (compressed air) discharged from the first auxiliary pipes 30a. The entire first guide member 32a is immersed in the culture solution L.

The first accommodation unit 12a is provided with a first temperature sensor 36a and a first light amount meter 38a. The first temperature sensor 36a detects the temperature of the culture solution L inside the first accommodation unit 12a. The first light amount meter 38a measures the amount of light incident on the first accommodation unit 12a.

The second accommodation unit 12b and the third accommodation unit 12c are both configured in the same manner as the first accommodation unit 12a. Specifically, the second accommodation unit 12b includes a second main pipe 28b, second auxiliary pipes 30b, and second guide members 32b. The second branch pipe 22b is connected to the second main pipe 28b. The second accommodation unit 12b is provided with a second temperature sensor 36b and a second light amount meter 38b. The third accommodation unit 12c includes a third main pipe 28c, third auxiliary pipes 30c, and third guide members 32c. The downstream end portion 23 of the flow pipe 20 is connected to the third main pipe 28c. The third accommodation unit 12c is provided with a third temperature sensor 36c and a third light amount meter 38c. The first temperature sensor 36a, the second temperature sensor 36b, and the third temperature sensor 36c are temperature detectors. The first light amount meter 38a, the second light amount meter 38b, and the third light amount meter 38c are light amount measurement devices.

The first temperature sensor 36a, the second temperature sensor 36b, and the third temperature sensor 36c are electrically connected to the control unit 18. The control unit 18 receives the temperatures detected by the first temperature sensor 36a, the second temperature sensor 36b, and the third temperature sensor 36c as information signals. Specifically, the temperature of the culture solution L in the first accommodation unit 12a, the temperature of the culture solution L in the second accommodation unit 12b, and the temperature of the culture solution L in the third accommodation unit 12c are input to the control unit 18.

The first light amount meter 38a, the second light amount meter 38b, and the third light amount meter 38c are electrically connected to the control unit 18. The control unit 18 receives the amounts of light measured by the first light amount meter 38a, the second light amount meter 38b, and the third light amount meter 38c as information signals. Specifically, the amount of light incident on the first accommodation unit 12a, the amount of light incident on the second accommodation unit 12b, and the amount of light incident on the third accommodation unit 12c are input to the control unit 18.

The control unit 18 includes a first time measurement circuit 40 and a second time measurement circuit 42. The first time measurement circuit 40 measures an elapsed time period from a gas supply starting step S1 to be described later or an elapsed time period from the end of a general step S5 to be described later. The second time measurement circuit 42 individually measures an elapsed time period from the start of an opening degree adjustment step S3 to be described later.

A threshold temperature which is an allowable lower limit value of the temperature, and a threshold amount of light which is an allowable lower limit value of the amount of light, are input in advance to the control unit 18. The control unit 18 compares the temperatures of the culture solution L detected by the first temperature sensor 36a to the third temperature sensor 36c with the threshold temperature. The control unit 18 compares the amounts of light measured by the first light amount meter 38a to the third light amount meter 38c with the threshold amount of light. Further, a predetermined time period is input to the control unit 18 in advance. The control unit 18 compares the elapsed time period measured by the first time measurement circuit 40 or the second time measurement circuit 42 with the predetermined time period.

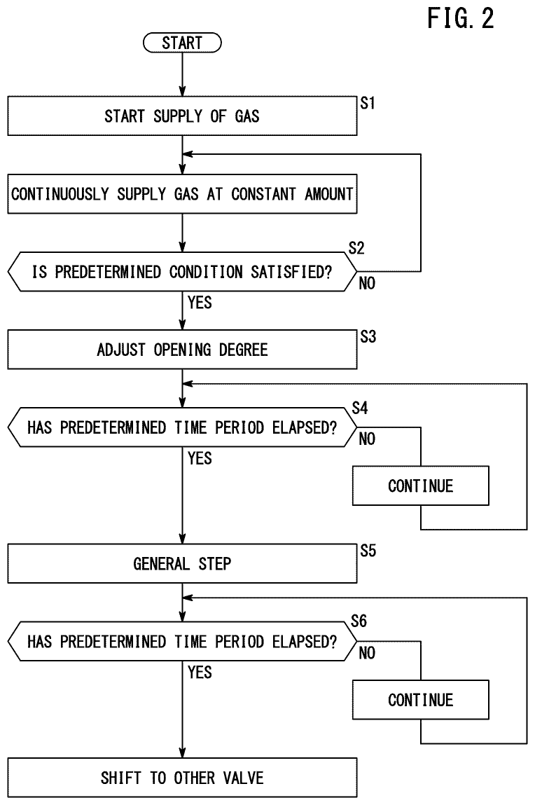

Next, with reference to the schematic process flow diagram shown in FIG. 2, a description will be given concerning the culture method according to the present embodiment. The culture method includes the gas supply starting step S1, a first determination step S2, the opening-degree adjustment step S3, a second determination step (agitation step) S4, the general step S5, and a third determination step S6. These steps S1 to S6 are executed based on sequence control executed by the control unit 18.

An operator inputs a command signal to “execute the gas supply starting step S1” to the control unit 18. Such a command signal is input to the control unit 18, for example, accompanying a switch provided in the control unit 18 being turned ON.

Upon receiving the command signal, the control unit 18 activates the air pump 16. Further, the control unit 18 adjusts the opening degrees of the first solenoid valve 24a, the second solenoid valve 24b, and the third solenoid valve 24c to, for example, about one third of full opening. The air pump 16 draws in and compresses the atmospheric air. Since the atmospheric air contains carbon dioxide, the atmospheric air compressed by the air pump 16 is a carbon dioxide-containing gas.

After being activated, the air pump 16 discharges the carbon dioxide-containing gas at a constant amount. That is, the air pump 16 is operated at a constant output in a rated operation mode. Hereinafter, this state is also referred to as “steady operation”.

Since the opening degrees of the first solenoid valve 24a, the second solenoid valve 24b, and the third solenoid valve 24c are the same, the carbon dioxide-containing gas flowing from the air pump 16 to the flow pipe 20 is substantially equally distributed to the first accommodation unit 12a, the second accommodation unit 12b, and the third accommodation unit 12c. That is, about one third of the carbon dioxide-containing gas discharged from the air pump 16 is distributed to the first branch pipe 22a and flows into the first main pipe 28a. Another about one third of the carbon dioxide-containing gas discharged from the air pump 16 is distributed to the second branch pipe 22b and flows into the second main pipe 28b. The remaining about one third of the carbon dioxide-containing gas discharged from the air pump 16 flows into the third main pipe 28c from the downstream end portion 23 of the flow pipe 20.

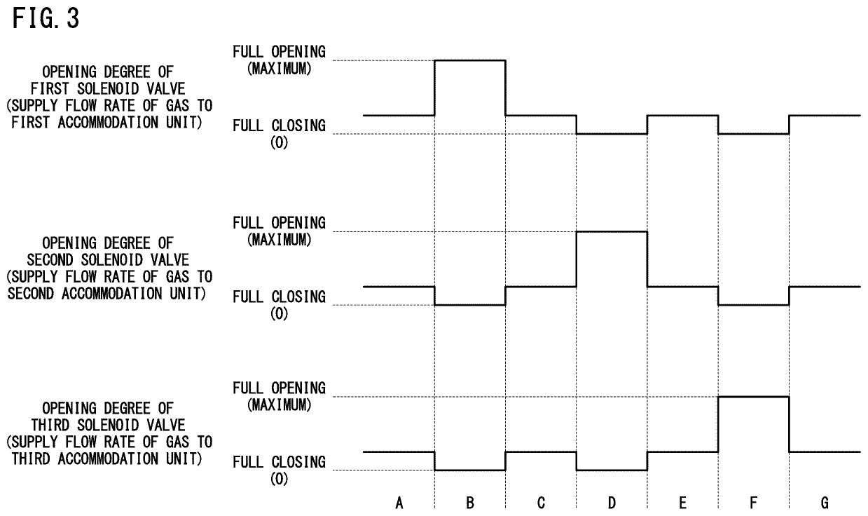

FIG. 3 is a graph showing changes in the opening degrees of the first solenoid valve 24a, the second solenoid valve 24b, and the third solenoid valve 24c, and changes in the supply flow rates of the carbon dioxide-containing gas supplied to the first main pipe 28a, the second main pipe 28b, and the third main pipe 28c. A region A in FIG. 3 represents a situation where the air pump 16 is operated in a steady state and the opening degrees of the first solenoid valve 24a, the second solenoid valve 24b, and the third solenoid valve 24c are the same.

In the first accommodation unit 12a, the carbon dioxide-containing gas flows into the culture solution L from the first branch pipe 22a through the first main pipe 28a and the first auxiliary pipes 30a, and then flows into the first guide members 32a. Similarly, in the second accommodation unit 12b, the carbon dioxide-containing gas flows into the culture solution L from the second branch pipe 22b through the second main pipe 28b and the second auxiliary pipes 30b, and then flows into the second guide members 32b. Similarly, in the third accommodation unit 12c, the carbon dioxide-containing gas flows into the culture solution L from the downstream end portion 23 of the flow pipe 20 through the third main pipe 28c and the third auxiliary pipes 30c, and then flows into the third guide members 32c.

In the first accommodation unit 12a to the third accommodation unit 12c, microalgae carry out photosynthesis using carbon dioxide in the carbon dioxide-containing gas. Consequently, microalgae are cultured in the first accommodation unit 12a to the third accommodation unit 12c. Through this culturing, carbon dioxide in the culture solution L is fixed in the microalgae. As can be understood from the above, based on the carbon dioxide being consumed by culturing the microalgae, it is possible to contribute to mitigating climate change or reducing its impact. In a case where phosphorus, nitrogen, potassium, and the like are added to the culture solution L, the microalgae take in such inorganic substances as nutrients.

The culture solution L and the microalgae are agitated in the first accommodation unit 12a to the third accommodation unit 12c by the carbon dioxide-containing gas supplied to the culture solution L. However, there is a tendency for the microalgae to settle over time. The microalgae also tend to settle when the temperature decreases or the amount of light decreases. In order to prevent the microalgae from settling, the control unit 18 performs control for temporarily supplying a large amount of carbon dioxide-containing gas to the first accommodation unit 12a to the third accommodation unit 12c. Hereinafter, a description will be given in detail concerning this feature.

The step in which culturing of the microalgae proceeds in a situation where the air pump 16 is operated in the steady state is a general step. During the general step, the first determination step S2 is executed. In the first determination step S2, the control unit 18 determines whether or not a predetermined condition set in advance in the control unit 18 is satisfied. In the present embodiment, the predetermined condition includes a first condition, a second condition, and a third condition described below. The first condition is that “a predetermined time period elapses after the execution of the gas supply starting step S1”. The second condition is that “the temperature of the culture solution L in each of the first accommodation unit 12a to the third accommodation unit 12c reaches the threshold temperature”. The third condition is that “the amount of light incident on each of the first accommodation unit 12a to the third accommodation unit 12c reaches the threshold amount of light”.

In order to determine the above, the temperatures detected by the first temperature sensor 36a to the third temperature sensor 36c, and the amounts of light measured by the first light amount meter 38a to third light amount meter 38c are input to the control unit 18. When the culture device 10 is installed outdoors, the light incident on the first accommodation unit 12a to the third accommodation unit 12c is sunlight. In the daytime, the temperature and the amount of light hardly differ greatly. Therefore, the temperature hardly reaches the threshold temperature, and the amount of light hardly reaches the threshold amount of light. That is, the second condition and the third condition are hardly satisfied in the daytime.

Meanwhile, the first time measurement circuit 40 of the control unit 18 measures an elapsed time period immediately after the execution of the gas supply starting step S1. When the elapsed time period reaches the predetermined time period input in advance to the control unit 18, the first condition is satisfied (“YES” in S2). In this case, the control unit 18 first executes the opening degree adjustment step S3, and then maintains a state in which the opening degrees of the first solenoid valve 24a to the third solenoid valve 24c have been adjusted. That is, the control unit 18 executes the second determination step S4. Thereafter, the control unit 18 executes the general step S5.

In the present embodiment, a case will be described as an example where the opening degree adjustment step S3 mainly targeted on the first solenoid valve 24a is executed first, the opening degree adjustment step S3 mainly targeted on the second solenoid valve 24b is executed next, and the opening degree adjustment step S3 mainly targeted on the third solenoid valve 24c is executed last. Here, the “opening degree adjustment mainly targeted on the first solenoid valve 24a” indicates that the opening degree of the first solenoid valve 24a is increased and the opening degrees of the second solenoid valve 24b and the third solenoid valve 24c are decreased in the opening degree adjustment step S3. The “opening degree adjustment mainly targeted on the second solenoid valve 24b” indicates that the opening degree of the second solenoid valve 24b is increased and the opening degrees of the first solenoid valve 24a and the third solenoid valve 24c are decreased in the opening degree adjustment step S3. The “opening degree adjustment mainly targeted on the third solenoid valve 24c” indicates that the opening degree of the third solenoid valve 24c is increased and the opening degrees of the first solenoid valve 24a and the second solenoid valve 24b are decreased in the opening degree adjustment step S3.

On the other hand, as described above, the general step S5 is a step of culturing microalgae under a situation where the air pump 16 is operated in the steady state and the opening degrees of the first solenoid valve 24a to the third solenoid valve 24c are not adjusted.

As described above, in the present embodiment, the opening degree adjustment mainly targeted on the first solenoid valve 24a, the opening degree adjustment mainly targeted on the second solenoid valve 24b, and the opening degree adjustment mainly targeted on the third solenoid valve 24c are executed in this order. In other words, the opening degree adjustment mainly targeted on the first solenoid valve 24a, the opening degree adjustment mainly targeted on the second solenoid valve 24b, and the opening degree adjustment mainly targeted on the third solenoid valve 24c are not simultaneously executed (do not overlap). In this manner, a time difference is provided between the opening degree adjustment mainly targeted on the first solenoid valve 24a and the opening degree adjustment mainly targeted on the second solenoid valve 24b. Similarly, a time difference is provided between the opening degree adjustment mainly targeted on the second solenoid valve 24b and the opening degree adjustment mainly targeted on the third solenoid valve 24c.

The opening degree adjustment mainly targeted on the first solenoid valve 24a will be described. In this case, when the elapsed time period from the execution of the gas supply starting step S1 reaches the predetermined time period, the control unit 18 makes the opening degree of the first solenoid valve 24a larger than the opening degree thereof up to this point. At the same time, the control unit 18 makes the opening degrees of the second solenoid valve 24b and the third solenoid valve 24c smaller than the opening degrees thereof up to this point. For example, the control unit 18 fully opens the first solenoid valve 24a and fully closes the second solenoid valve 24b and the third solenoid valve 24c. As a result, the entire amount of the carbon dioxide-containing gas from the air pump 16 is supplied to the first accommodation unit 12a. In contrast, the supply flow rate of the carbon dioxide-containing gas supplied to the second accommodation unit 12b and the third accommodation unit 12c is 0. This situation is represented by a region B of FIG. 3.

The second time measurement circuit 42 of the control unit 18 measures an elapsed time period immediately after the execution of the opening degree adjustment step S3 mainly targeted on the first solenoid valve 24a. During this measurement, the second determination step S4 is executed. Specifically, the control unit 18 determines whether or not the elapsed time period from the execution of the opening degree adjustment step S3 mainly targeted on the first solenoid valve 24a reaches a predetermined time period input in advance to the control unit 18.

When the elapsed time period reaches the predetermined time period (“YES” in S4), the control unit 18 shifts the culture method to the general step S5. Specifically, when the elapsed time period from the execution of the opening degree adjustment step S3 reaches the predetermined time period, the control unit 18 makes the opening degree of the first solenoid valve 24a smaller than the opening degree thereof at the time of the opening degree adjustment step S3. At the same time, the control unit 18 makes the opening degrees of the second solenoid valve 24b and the third solenoid valve 24c larger than the opening degrees thereof at the time of the opening degree adjustment step S3. For example, the control unit 18 sets the first solenoid valve 24a to the original opening degree (one third of the full opening) and sets the second solenoid valve 24b and the third solenoid valve 24c to the original opening degrees (one third of the full opening). As a result, the carbon dioxide-containing gas from the air pump 16 is substantially equally distributed to the first accommodation unit 12a to the third accommodation unit 12c in the same manner as described above. This situation is represented by a region C of FIG. 3. As the predetermined time period, for example, an appropriate time period between several tens of seconds and several minutes is selected. The same applies to the following.

In this manner, a large amount of carbon dioxide-containing gas is supplied to the first accommodation unit 12a during a period from the execution of the opening degree adjustment step S3 to the transition to the general step S5 (during the period in which the second determination step S4 is executed). The culture solution L in the first accommodation unit 12a is agitated by the large amount of carbon dioxide-containing gas. Therefore, the microalgae in the culture solution L are also agitated. In this manner, the second determination step S4 serves as an agitation step of strongly agitating the culture solution L and the microalgae.

Since the culture solution L and the microalgae are strongly agitated, settling or flocculation of the microalgae in the first accommodation unit 12a is avoided. Therefore, light is substantially evenly incident on the microalgae in the first accommodation unit 12a. Consequently, the microalgae in the first accommodation unit 12a actively perform photosynthesis and sufficiently fix carbon dioxide. As a result, a sufficient amount of carbon dioxide can be consumed.

In the third determination step S6, the first time measurement circuit 40 of the control unit 18 measures an elapsed time period immediately after the execution of the general step S5. When the elapsed time period reaches a predetermined time period input in advance to the control unit 18 (“YES” in S6), the control unit 18 executes the opening degree adjustment step S3 mainly targeted on the second solenoid valve 24b.

Specifically, when the elapsed time period from the execution of the general step S5 reaches the predetermined time period, the control unit 18 makes the opening degree of the second solenoid valve 24b larger than the opening degree thereof up to this point. At the same time, the control unit 18 makes the opening degrees of the first solenoid valve 24a and the third solenoid valve 24c smaller than the opening degrees thereof up to this point. For example, the control unit 18 fully opens the second solenoid valve 24b and fully closes the first solenoid valve 24a and the third solenoid valve 24c. As a result, the entire amount of the carbon dioxide-containing gas from the air pump 16 is supplied to the second accommodation unit 12b. In contrast, the supply flow rate of the carbon dioxide-containing gas supplied to the first accommodation unit 12a and the third accommodation unit 12c is 0. This situation is represented by a region D of FIG. 3.

The second time measurement circuit 42 of the control unit 18 measures an elapsed time period immediately after the execution of the opening degree adjustment step S3 mainly targeted on the second solenoid valve 24b. During this measurement, the second determination step S4 is executed. Specifically, the control unit 18 determines whether or not the elapsed time period from the execution of the opening degree adjustment step S3 mainly targeted on the second solenoid valve 24b reaches a predetermined time period input in advance to the control unit 18.

When the elapsed time period reaches the predetermined time period, the control unit 18 shifts the culture method to the general step S5. Specifically, when the elapsed time period from the execution of the opening degree adjustment step S3 mainly targeted on the second solenoid valve 24b reaches the predetermined time period, the control unit 18 makes the opening degree of the second solenoid valve 24b smaller than the opening degree thereof at the time of the opening degree adjustment step S3. At the same time, the control unit 18 makes the opening degrees of the first solenoid valve 24a and the third solenoid valve 24c larger than the opening degrees thereof at the time of the opening degree adjustment step S3. For example, the control unit 18 sets the second solenoid valve 24b to the original opening degree (one third of the full opening) and sets the first solenoid valve 24a and the third solenoid valve 24c to the original opening degrees (one third of the full opening). As a result, the carbon dioxide-containing gas from the air pump 16 is substantially equally distributed to the first accommodation unit 12a to the third accommodation unit 12c in the same manner as described above. This situation is represented by a region E of FIG. 3.

A large amount of carbon dioxide-containing gas is supplied to the second accommodation unit 12b during a period from when the opening degree adjustment step S3 mainly targeted on the second solenoid valve 24b is executed until when the general step S5 is executed. The culture solution L in the second accommodation unit 12b is agitated by the large amount of carbon dioxide-containing gas. Therefore, the microalgae in the culture solution L are also agitated. For this reason, settling or flocculation of the microalgae in the second accommodation unit 12b is avoided. Therefore, light is substantially evenly incident on the microalgae in the second accommodation unit 12b. Consequently, the microalgae in the second accommodation unit 12b actively perform photosynthesis and sufficiently fix carbon dioxide. As a result, a sufficient amount of carbon dioxide can be consumed.

The first time measurement circuit 40 of the control unit 18 measures an elapsed time period immediately after the execution of the general step S5. When the elapsed time period reaches a predetermined time period input in advance to the control unit 18, the control unit 18 executes the opening degree adjustment step S3 mainly targeted on the third solenoid valve 24c.

Specifically, when the elapsed time period from the execution of the general step S5 reaches the predetermined time period, the control unit 18 makes the opening degree of the third solenoid valve 24c larger than the opening degree thereof up to this point. At the same time, the control unit 18 makes the opening degrees of the first solenoid valve 24a and the second solenoid valve 24b smaller than the opening degrees thereof up to this point. For example, the control unit 18 fully opens the third solenoid valve 24c and fully closes the first solenoid valve 24a and the second solenoid valve 24b. As a result, the entire amount of the carbon dioxide-containing gas from the air pump 16 is supplied to the third accommodation unit 12c. In contrast, the supply flow rate of the carbon dioxide-containing gas supplied to the first accommodation unit 12a and the second accommodation unit 12b is 0. This situation is represented by a region F of FIG. 3.

The second time measurement circuit 42 of the control unit 18 measures an elapsed time period immediately after the execution of the opening degree adjustment step S3 mainly targeted on the third solenoid valve 24c. During this measurement, the second determination step S4 is executed. Specifically, the control unit 18 determines whether or not the elapsed time period from the execution of the opening degree adjustment step S3 mainly targeted on the third solenoid valve 24c reaches a predetermined time period input in advance to the control unit 18.

When the elapsed time period reaches the predetermined time period, the control unit 18 shifts the culture method to the general step S5. Specifically, when the elapsed time period from the execution of the opening degree adjustment step S3 mainly targeted on the third solenoid valve 24c reaches the predetermined time period, the control unit 18 makes the opening degree of the third solenoid valve 24c smaller than the opening degree thereof at the time of the opening degree adjustment step S3. At the same time, the control unit 18 makes the opening degrees of the first solenoid valve 24a and the second solenoid valve 24b larger than the opening degrees thereof at the time of the opening degree adjustment step S3. For example, the control unit 18 sets the third solenoid valve 24c to the original opening degree (one third of the full opening) and sets the first solenoid valve 24a and the second solenoid valve 24b to the original opening degrees (one third of the full opening). As a result, the carbon dioxide-containing gas from the air pump 16 is substantially equally distributed to the first accommodation unit 12a to the third accommodation unit 12c in the same manner as described above. This situation is represented by a region G of FIG. 3.

A large amount of carbon dioxide-containing gas is supplied to the third accommodation unit 12c during a period from when the opening degree adjustment step S3 mainly targeted on the third solenoid valve 24c is executed until when the general step S5 is executed. The culture solution L in the third accommodation unit 12c is agitated by the large amount of carbon dioxide-containing gas. Therefore, the microalgae in the culture solution L are also agitated. For this reason, settling or flocculation of the microalgae in the third accommodation unit 12c is avoided. Therefore, light is substantially evenly incident on the microalgae in the third accommodation unit 12c. Consequently, the microalgae in the third accommodation unit 12c actively perform photosynthesis and sufficiently fix carbon dioxide. As a result, a sufficient amount of carbon dioxide can be consumed.

As described above, microalgae can be favorably cultured in the first accommodation unit 12a to the third accommodation unit 12c.

At night, the temperature of the culture solution L is lower than that in the daytime, and the amount of light incident on each of the first accommodation unit 12a to the third accommodation unit 12c decreases. Under such a situation, the second condition or the third condition may be satisfied before the first condition is satisfied. In this case as well, in the same manner as described above, the control unit 18 sequentially executes the opening degree adjustment mainly targeted on the first solenoid valve 24a, the opening degree adjustment mainly targeted on the second solenoid valve 24b, and the opening degree adjustment mainly targeted on the third solenoid valve 24c. Therefore, even under a situation in which microalgae tend to settle in a relatively short time, such as at night, microalgae can be favorably cultured in the first accommodation unit 12a to the third accommodation unit 12c.

In this manner, according to the present embodiment, a large amount of carbon dioxide-containing gas can be temporarily supplied to the culture solution L by executing the opening degree adjustment step S3. Therefore, while microalgae are cultured in the manner as described above, the air pump 16 is maintained in steady operation. That is, even when a large amount of carbon dioxide-containing gas is supplied to the culture solution L in order to agitate microalgae, it is not necessary to increase the carbon dioxide-containing gas discharge amount of the air pump 16. For the above reason, it is not necessary to select the air pump 16 having an excessive capacity for discharging a large amount of carbon dioxide-containing gas.

In other words, according to the present embodiment, it is possible to select the air pump 16 having an appropriate capacity for discharging the carbon dioxide-containing gas in an amount sufficient for culturing microalgae. Therefore, an increase in electric power consumption per unit time period is avoided. This can reduce the running cost in culturing microalgae.

As described above, the present embodiment discloses the culture method for culturing microalgae in the culture device (10) including the plurality of accommodation units (12a to 12c) configured to accommodate the culture solution (L) and the microalgae, the gas supply unit (16) configured to supply a gas to the plurality of accommodation units, and the plurality of gas supply lines (20, 22a, 22b) configured to connect the gas supply unit with the plurality of accommodation units, respectively, wherein the plurality of gas supply lines are provided with the plurality of valves (24a to 24c), respectively, the culture method including: the opening degree adjustment step (S3) of adjusting the opening degree of at least one of the plurality of valves during culturing of the microalgae in the plurality of accommodation units; and an agitation step (S4) of culturing the microalgae in a state in which the opening degree of the at least one of the plurality of valves is adjusted, wherein, in the opening degree adjustment step, by adjusting the opening degree of the at least one of the plurality of valves, the supply of the gas to at least one accommodation unit of the plurality of accommodation units is stopped or the supply flow rate of the gas supplied to the at least one accommodation unit is decreased, and the supply flow rate of the gas supplied to at least one remaining accommodation unit is increased.

The present embodiment discloses the culture device (10) that cultures microalgae in the culture solution (L), the culture device including: the plurality of accommodation units (12a to 12c) configured to accommodate the culture solution and the microalgae; the gas supply unit (16) configured to supply a gas to the plurality of accommodation units; the plurality of gas supply lines (20, 22a, 22b) configured to connect the gas supply unit with the plurality of accommodation units, respectively; the plurality of valves (24a to 24c) provided in the plurality of gas supply lines, respectively; and the control unit (18) configured to individually adjust the opening degree of each of the plurality of valves, wherein, by adjusting the opening degree of at least one of the plurality of valves during culturing of the microalgae in the plurality of accommodation units, the control unit stops the supply of the gas to at least one accommodation unit of the plurality of accommodation units or decreases the supply flow rate of the gas supplied to the at least one accommodation unit, and increases the supply flow rate of the gas supplied to at least one remaining accommodation unit.

By adjusting the opening degree, the supply of the gas is stopped, or the supply flow rate is decreased in at least one of the plurality of accommodation units. In contrast, the supply flow rate of the gas is increased in at least one remaining accommodation unit. That is, the supply flow rate of the gas is adjusted.

Therefore, even if the supply flow rate of the gas from the gas supply unit is constant, a large amount of gas can be temporarily supplied to at least one of the plurality of accommodation units. Therefore, it is not necessary to select a gas supply unit having an excessive capacity.

That is, in the present embodiment, it is possible to use a gas supply unit that consumes a small amount of electric power per unit time period. Therefore, in performing culturing, it is possible to reduce the running cost.

In the accommodation unit in which the supply flow rate of the gas is temporarily increased, the microalgae and the culture solution are agitated. Therefore, settling or flocculation of the microalgae is suppressed. Accordingly, the light is incident on the microalgae substantially evenly. Further, since the culture solution is agitated, carbon dioxide becomes diffused throughout the entirety of the accommodation unit. Due to this reason, the microalgae actively carry out photosynthesis. Since a large amount of carbon dioxide becomes fixed in the microalgae by way of photosynthesis, such a feature can be expected to contribute to mitigating climate change or reducing its impact.

The present embodiment discloses the culture method including a plurality of opening degree adjustment steps including the opening degree adjustment step, wherein the supply flow rate of the gas is increased in all of the plurality of accommodation units by performing the plurality of opening degree adjustment steps.

The present embodiment discloses the culture device wherein the control unit performs a plurality of times of opening degree adjustments including the adjusting of the opening degree performed on the at least one of the plurality of valves, and increases the supply flow rate of the gas in all of the plurality of accommodation units by performing the plurality of times of opening degree adjustments.

According to this feature, the supply flow rate of the gas supplied to all of the accommodation units can be sequentially and temporarily increased. Therefore, even if the supply flow rate of the gas from the gas supply unit is constant, it is possible to prevent the microalgae from settling or flocculating in all of the plurality of accommodation units. As a result, the microalgae can actively perform photosynthesis in all the accommodation units.

The present embodiment discloses the culture method wherein the plurality of opening degree adjustment steps for the plurality of accommodation units do not overlap each other.

The present embodiment discloses the culture device wherein the control unit performs the plurality of times of opening degree adjustments for the plurality of accommodation units without causing the opening degree adjustments to overlap each other.

In this case, the supply flow rates of the gas supplied to the plurality of accommodation units are prevented from being simultaneously adjusted. Therefore, the gas can be supplied at a sufficient supply flow rate to the accommodation unit in which the culture solution needs to be agitated.

The present embodiment discloses the culture method wherein, in the opening degree adjustment step, the opening degree of the at least one of the plurality of valves is set to full opening, and the opening degree of at least one remaining valve is set to full closing.

The present embodiment discloses the culture device wherein the control unit sets the opening degree of the at least one of the plurality of valves to full opening and sets the opening degree of at least one remaining valve to full closing.

In this case, the gas is not distributed to the accommodation unit provided with the valve which has been fully closed. On the other hand, a large amount of gas is distributed to the accommodation unit provided with the valve which has been fully opened. Therefore, a larger amount of gas can be supplied to the accommodation unit in which the culture solution needs to be agitated.

The present embodiment discloses the culture method wherein the opening degree adjustment step is executed when a predetermined condition set in advance is satisfied.

The present embodiment discloses the culture device wherein the control unit adjusts the opening degree of the at least one of the plurality of valves when a predetermined condition set in advance is satisfied.

The predetermined condition is, for example, a condition under which microalgae tend to settle. Since the opening degree adjustment is executed when such a situation occurs, it is possible to prevent the microalgae from settling or flocculating.

Even in a case where a gas sufficient for culturing microalgae is continuously supplied to the accommodation units, there is a tendency for the microalgae to settle over time. In order to avoid such a situation, it is preferable to execute the opening degree adjustment and the second opening degree adjustment when the predetermined time period has elapsed. That is, the present embodiment discloses the culture method wherein the predetermined condition is that the predetermined time period has elapsed.

The present embodiment discloses the culture device wherein the predetermined condition is elapse of the predetermined time period, and the control unit adjusts the opening degree of the at least one of the plurality of valves when the predetermined time period has elapsed.

Microalgae also tend to settle when the surrounding environmental temperature decreases. In order to avoid this situation, it is preferable to execute the opening degree adjustment and the second opening degree adjustment when the temperature of the culture solution reaches the allowable lower limit value. That is, the present embodiment discloses the culture method wherein the predetermined condition is that the temperature of the culture solution reaches the threshold temperature or lower.

The present embodiment discloses the culture device further including the temperature detector (36a to 36c) configured to measure the temperature of the culture solution, wherein the predetermined condition is that the temperature reaches the threshold temperature or lower, and the control unit adjusts the opening degree of the at least one of the plurality of valves when the temperature reaches the threshold temperature or lower.

Microalgae also tend to settle when the amount of light incident on the accommodation units decreases. In order to avoid this situation, it is preferable to execute the opening degree adjustment and the second opening degree adjustment when the amount of light reaches the allowable lower limit value. That is, the present embodiment discloses the culture method wherein the predetermined condition is that the amount of light emitted to each of the plurality of accommodation units reaches the threshold amount of light or less.

The present embodiment discloses the culture device further including the light amount measurement device (38a to 38c) configured to measure the amount of light emitted to each of the plurality of accommodation units, wherein the predetermined condition is that the amount of light reaches the threshold amount of light or less, and the control unit adjusts the opening degree of the at least one of the plurality of valves when the amount of light reaches the threshold amount of light or less.

The present embodiment discloses the culture method wherein the pump (16) is used as the gas supply unit, and the output of the pump is set to be constant both in a case where the opening degree adjustment step is performed and in a case where the general step (S5) of supplying the gas to all of the plurality of accommodation units without performing the opening degree adjustment step is performed.

The present embodiment discloses the culture device wherein the gas supply unit is the pump (16), and the pump is operated at a constant output both in a case where adjustment of the opening degree of the at least one of the plurality of valves is performed and in a case where the gas is supplied to all of the plurality of accommodation units without performing the adjustment.

As described above, according to the present embodiment, even when the gas supply flow rate from the gas supply unit such as a pump is constant, a large amount of carbon dioxide-containing gas can be temporarily supplied to a predetermined accommodation unit. That is, even when a large amount of carbon dioxide-containing gas is supplied to the culture solution in order to agitate the microalgae, the pump can be operated at a constant output.

In other words, in this case, when a large amount of carbon dioxide-containing gas is temporarily supplied to one accommodation unit, it is not necessary to increase the discharge amount of the pump. Therefore, it is not necessary to select a pump having an excessive capacity for discharging a large amount of carbon dioxide-containing gas. In other words, it is possible to select a pump having an appropriate capacity for discharging a sufficient amount of gas for culturing the microalgae. Therefore, an increase in electric power consumption per unit time period is avoided. This can reduce the running cost in culturing microalgae.

The present invention is not limited to the above disclosure, and various modifications are possible without departing from the essence and gist of the present invention.

For example, the number of the accommodation units is not limited to three. The number of the accommodation units may be two or may be four or more. The number of the gas supply lines and the number of the valves are determined according to the number of the accommodation units.

In the opening degree adjustment step S3, it is sufficient that the opening degree of the main target valve is larger than the opening degree of the valve other than the main target valve. In other words, the opening degree of each valve is not particularly limited. For example, the opening degree of the main target valve (the valve whose opening degree is increased) is not limited to full opening. Similarly, the opening degree of the valve other than the main target valve (the valve whose opening degree is reduced) is not limited to full closing.
What is claim is:

1. A culture method for culturing microalgae in a culture device including a plurality of accommodation units configured to accommodate a culture solution and the microalgae, a gas supply unit configured to supply a gas to the plurality of accommodation units, and a plurality of gas supply lines configured to connect the gas supply unit with the plurality of accommodation units, respectively,
wherein the plurality of gas supply lines are provided with a plurality of valves, respectively,
the culture method comprising:
an opening degree adjustment step of adjusting an opening degree of at least one of the plurality of valves during culturing of the microalgae in the plurality of accommodation units; and
an agitation step of culturing the microalgae in a state in which the opening degree of the at least one of the plurality of valves is adjusted,
wherein, in the opening degree adjustment step, by adjusting the opening degree of the at least one of the plurality of valves, supply of the gas to at least one accommodation unit of the plurality of accommodation units is stopped or a supply flow rate of the gas supplied to the at least one accommodation unit is decreased, and a supply flow rate of the gas supplied to at least one remaining accommodation unit is increased.

2. The culture method according to claim 1, comprising a plurality of opening degree adjustment steps including the opening degree adjustment step, wherein
the supply flow rate of the gas is increased in all of the plurality of accommodation units by performing the plurality of opening degree adjustment steps.

3. The culture method according to claim 2, wherein
the plurality of opening degree adjustment steps for the plurality of accommodation units do not overlap each other.

4. The culture method according to claim 1, wherein
in the opening degree adjustment step, the opening degree of the at least one of the plurality of valves is set to full opening, and an opening degree of at least one remaining valve is set to full closing.

5. The culture method according to claim 1, wherein
the opening degree adjustment step is executed when a predetermined condition set in advance is satisfied.

6. The culture method according to claim 5, wherein
the predetermined condition is that a predetermined time period has elapsed.

7. The culture method according to claim 5, wherein
the predetermined condition is that a temperature of the culture solution reaches a threshold temperature or lower.

8. The culture method according to claim 5, wherein
the predetermined condition is that an amount of light emitted to each of the plurality of accommodation units reaches a threshold amount of light or less.

9. The culture method according to claim 1, wherein
a pump is used as the gas supply unit, and an output of the pump is set to be constant both in a case where the opening degree adjustment step is performed and in a case where a general step of supplying the gas to all of the plurality of accommodation units without performing the opening degree adjustment step is performed.

10. A culture device that cultures microalgae in a culture solution, the culture device comprising:
a plurality of accommodation units configured to accommodate the culture solution and the microalgae;
a gas supply unit configured to supply a gas to the plurality of accommodation units;
a plurality of gas supply lines configured to connect the gas supply unit with the plurality of accommodation units, respectively;
a plurality of valves provided in the plurality of gas supply lines, respectively; and
a control unit configured to individually adjust an opening degree of each of the plurality of valves,
wherein, by adjusting the opening degree of at least one of the plurality of valves during culturing of the microalgae in the plurality of accommodation units, the control unit stops supply of the gas to at least one accommodation unit of the plurality of accommodation units or decreases a supply flow rate of the gas supplied to the at least one accommodation unit, and increases a supply flow rate of the gas supplied to at least one remaining accommodation unit.

11. The culture device according to claim 10, wherein
the control unit performs a plurality of times of opening degree adjustments including the adjusting of the opening degree performed on the at least one of the plurality of valves, and
the control unit increases the supply flow rate of the gas in all of the plurality of accommodation units by performing the plurality of times of opening degree adjustments.

12. The culture device according to claim 11, wherein
the control unit performs the plurality of times of opening degree adjustments for the plurality of accommodation units without causing the opening degree adjustments to overlap each other.

13. The culture device according to claim 10, wherein
the control unit sets the opening degree of the at least one of the plurality of valves to full opening, and sets an opening degree of at least one remaining valve to full closing.

14. The culture device according to claim 10, wherein
the control unit adjusts the opening degree of the at least one of the plurality of valves when a predetermined condition set in advance is satisfied.

15. The culture device according to claim 14, wherein
the predetermined condition is elapse of a predetermined time period, and the control unit adjusts the opening degree of the at least one of the plurality of valves when the predetermined time period has elapsed.

16. The culture device according to claim 14, further comprising a temperature detector configured to measure a temperature of the culture solution, wherein
the predetermined condition is that the temperature reaches a threshold temperature or lower, and the control unit adjusts the opening degree of the at least one of the plurality of valves when the temperature reaches the threshold temperature or lower.

17. The culture device according to claim 14, further comprising a light amount measurement device configured to measure an amount of light emitted to each of the plurality of accommodation units, wherein
the predetermined condition is that the amount of light reaches a threshold amount of light or less, and the control unit adjusts the opening degree of the at least one of the plurality of valves when the amount of light reaches the threshold amount of light or less.

18. The culture device according to claim 10, wherein
the gas supply unit is a pump, and the pump is operated at a constant output both in a case where adjustment of the opening degree of the at least one of the plurality of valves is performed and in a case where the gas is supplied to all of the plurality of accommodation units without performing the adjustment.
